(19) United States
(12) Patent Application Publication
(10) Pub. No.: US 2024/0047077 A1
(43) Pub. Date: Feb. 8, 2024

(54) METHOD AND SYSTEM FOR GENERATING PERSONALIZED BIOLOGICAL AGE PREDICTION MODEL

(71) Applicant: YOOJINBIOSOFT CO., LTD., Goyang-si (KR)

(72) Inventors: Jin Tae YOU, Seoul (KR); Jin Ho YOO, Goyang-si (KR)

(21) Appl. No.: 18/259,054

(22) PCT Filed: Feb. 24, 2022
(86) PCT No.: PCT/KR2022/002749

§ 371 Date: Jun. 22, 2023

(30) Foreign Application Priority Data
Aug. 28, 2021 (KR) 10-2021-0114310

(51) Int. Cl. G16H 50/70

(52) U.S. Cl. CPC G16H 50/70

(57) ABSTRACT

A method and a system thereof for generating a personalized biological age prediction model are proposed. The method and the system generate a model capable of predicting a biological age for each individual by obtaining an excess age for a chronological age for each age on the basis of medical checkup data. More particularly, the method and the system build a biological age prediction model by gender and chronological age group in consideration of aging mechanisms different from each other according to the gender and the chronological age group, and enable predicting the biological age according to the biological age prediction model for each age group.

### TECHNICAL FIELD

The present disclosure relates to a method for generating a model for predicting a personalized biological age and, more particularly, to a method and a system thereof for generating a personalized biological age prediction model capable of predicting a biological age for each individual by obtaining an excess age for a chronological age by age on the basis of medical checkup data.

### BACKGROUND ART

In general, a chronological age indicates a difference between a current year and a year of birth, and regardless of an individual's current health conditions, all people born in the same year are meant to have the same chronological age, naturally.

Accordingly, since “aging” related to the individual's current health conditions or overall declines in physical functions may not be fully expressed only by his or her chronological age, it is required to develop technology that may predict or measure “biological age” indicating the declines in the physical functions related to the aging.

Unlike chronological age, biological age is a numerical value of parts that vary depending on the overall health conditions of the body. That is, degrees of health and aging of the body are expressed in the numerical value.

Since even people of the same chronological age may have different health conditions, it may be said that rather than using chronological age, using biological age obtained by measuring or estimating the overall health conditions of the body is more accurate to measure current overall health conditions, aging, and further, actual life expectancy.

### Existing Research for Predicting/Measuring Biological Age

Research to measure biological age began with Comfort in 1969 and has been steadily conducted until now.

Factors required for biomarkers used to measure biological age are listed such as
1) providing information about the body's functions or metabolic system,
2) possessing quantitative characteristics that correlate with chronological age,
3) possessing reproducibility, sensitivity, and specific characteristics, and
4) having suitability for application not only to humans but also to laboratory animals.

Taking into account such factors, research has been conducted to measure biological age by using physical, physiological, and biochemical biomarkers.

Biomarkers commonly used to measure biological age include body mass index (BMI), blood pressure (i.e., systolic blood pressure and diastolic blood pressure), waist circumference, lung capacity, muscle mass, albumin, cholesterol levels, etc. Biological age measurement models are being studied by applying multivariable linear regression analysis and principal component analysis (PCA), which use these biomarkers as independent factors.

### Research on Mortality Risk Prediction

Levine and Crimmins conducted a study to predict mortality rates for 10 years by using biological age, and Brown and McDaid conducted investigation and research on influences on adult mortality rates affected by factors, such as chronological age, education levels, gender, income, marital status, occupations, race, religion, smoking, drinking, physical activity levels, obesity, etc.

Meanwhile, there is also a case of a study using a model in which a logistic regression model was created with nine factors including gender, whether a person smokes or not, chronological age, and life insurance underwriting class to evaluate the risk of death.

In South Korea, there is a case of creating a biological age measurement model by using medical checkup data for a large number of Korean people and then using a Cox regression model to study influences on seventeen-year survival and mortality in a case where biological age is measured higher than chronological age.

In a biological age measurement model currently published in a form of papers or patents, only one numerical value is presented such that an individual's biological age is equal to 55.7 years. Since quantitative and qualitative interpretations of what this number means are not objective and are unclear, the individual's aging status is required to be expressed in a different form, such as a biological age probability spectrum/distribution, rather than a single numerical value.

### SCI-level Papers Related to Biological Age Measurement

Currently published biological age measurement models
(a) A new approach to the concept and computation of biological age
2006, Mechanisms of Aging and Development (conducted on Czech test subjects)
non-linearly modeling of biomarker influences
(b) A method for identifying biomarkers of aging and constructing an index of biological age in humans.
2007, Journal of Gerontology (Kyoto University, conducted on Japanese male test subjects)
modeling using PCA analysis technique (R2=0.52)
(c) Development of models for predicting biological age (BA) with physical, biochemical, and hormonal parameters
2008, Arch Gerontol Geriatr. (classifying total body, physical, biochemistry, and hormonal age, conducted on Korean test subjects)
modeling using multiple linear regression (male R2=0.62, female R2=0.66)
(d) Developing a biological age assessment equation using principal component analysis and clinical biomarkers of aging in Korean men
2009, Archives of Gerontology and Geriatrics (classifying normal, hyperglycemic, and diabetic patients by age group, Seoul National University, conducted on Korean male test subjects)
modeling using PCA analysis technique (R2=0.581)
(e) Development and Application of Biological Age Prediction Models with Physical Fitness and Physiological Components in Korean Adults
2012, Gerontology (Classifying normal and obese patients by age group, Asan Medical Center, conducted on Korean test subjects)
modeling using PCA analysis technique (male R2=0.638, female R2=0.672)
(f) Analyzing influence of biological age on death
Biological age as a useful index to predict seventeen-year survival and mortality in Koreans
2017, BMC Geriatrics (analyzing the influence of biological age on death by using data from 17-year follow-up of 550,000 Koreans)

Here, R2 above means a coefficient of determination.

### Multivariable Linear Regression Analysis Model: MLR

FIG. 3 is a view illustrating a linear regression line.

The linear regression line in FIG. 3 can be expressed as a linear regression equation such as Y=a+b*X.

The points shown in FIG. 3 represent the measured coordinates X (i.e., checkup numerical values) and Y (i.e., age) of each individual. As the checkup numerical values increase, chronological age tends to increase. When these points are expressed as a linear regression model, an effect is expressed such that the higher checkup numerical values, the greater the age.

(The quantitative influence of the checkup numerical values on the increase in age is expressed as a slope of a linear regression equation)

That is, an outline of the biological age prediction model using the linear regression model may be summarized such that biological age, which is estimated to exist somewhere in an increase or decrease relationship between the checkup numerical values and the age (i.e., more precisely, the chronological age), is considered as a Y value of the linear regression equation above.

A multivariable linear regression analysis model can be expressed as in Equation 1 below.

Y=a0+a1×BMI+a2×SBP+a3×HDL [Equation 1]

### Multivariable Linear Regression (MLR) Model

The above Equation 1 shows a linear influence of independent variables on chronological age by setting the chronological age as a dependent variable Y and three variables of BMI, SBP, and HDL as independent variables.

Here, a1, a2, and a3 are regression coefficients, and represent the respective influences of BMI, SBP, and HDL on chronological age.

In addition, a0 is an intercept or a regression constant.

Y calculated through Equation 1 is a value that is calculated when measurement values of BMI, SBP, and HDL are input, and the key to the MLR model is to regard this value as biological age.

Such a multivariable linear regression (MLR) model has the following problems.

In a case of a young person, his or her biological age BA is predicted to be overestimated compared to his or her chronological age CA, whereas in a case of an older person, his or her biological age BA is predicted to be underestimated.

This result is presumed to be due to characteristics of data, and exactly what mechanism is responsible is unknown.

FIG. 4 is a graph illustrating a relationship between chronological age X and biological age Y, and shows an example of overestimation and underestimation of a multivariable linear regression model.

In biological age BA, a contradiction exists in that chronological age CA is dependent (i.e., a dependent variable) on medical checkup items.

That is, the chronological age CA is not a medical checkup item, but dependent on calendar time.

In particular, when a correlation between a medical checkup item and the chronological age CA is “1”, the medical checkup item itself is useless. (Basis: Ingram, 1988)

This means that there is a contradiction in an assumption itself established when a model is built.

Papers that mentioned the problems of multivariable linear regression model are as follows:
(a) 2008, Linear regression model—MLR model
Development of models for predicting biological age BA with physical, biochemical, and hormonal parameters
(b) 2009, Seoul National University Hospital model—PCA model
Developing a biological age assessment equation using principal component analysis and clinical biomarkers of aging in Korean
(c) 2011, Asan Hospital model—PCA model
Development and Application of Biological Age Prediction Models with Physical Fitness and Physiological Components in Korean Adults
(d) 2010, Paper comparing between biological age models
An empirical comparative study on biological age estimation algorithms with an application of Work Ability Index (WAI)

### Description of Principal Component Analysis Model, PCA

Principal Component Analysis (PCA) is a method operating as follows:

As shown in FIG. 5, the method analyzes common characteristics represented by multiple variables v1 to v5 to find a small number of independent factors (i.e., factor 1 and factor 2) that may represent the common characteristics.

For example, two independent factors, respectively called “a blood pressure factor” and “a cholesterol factor”, may be extracted when PCA analysis is performed by using five variables such as SBP, DBP, HDL, LDL, and TG.

PCA is applied to a plurality of medical checkup variables, such as BMI, WST, SBP, DBP, AST, ALT, GGTP, HDL, LDL, TG, and lung capacity, so that “one factor” common to these variables is extracted.

In this way, it is analyzed that “the chronological age and one factor extracted through PCA have a significant level of positive correlation therebetween”. (Pearson' correlation coefficient 0.8)

Accordingly, the key point of the PCA biological age prediction model is to determine “one factor” extracted by the PCA method as “biological age” representing actual aging status of a person.

The following are biological age prediction models using PCA.
(a) 2009, Seoul National University Hospital model—PCA model,
Developing a biological age assessment equation using principal component analysis and clinical biomarkers of aging in Korean men
(b) 2011, Asan Hospital model—PCA model
Development and Application of Biological Age Prediction Models with Physical Fitness and Physiological Components in Korean Adults
(c) 2007, Japanese model—PCA model
A Method for Identifying Biomarkers of Aging and Constructing an Index of Biological Age in Humans—PCA

### Characteristics of Biological Age Prediction Model Using PCA

In PCA analysis, unlike multivariable regression analysis, there is no distinction between dependent and independent variables. That is, when the number of medical checkup items is five, the PCA analysis may be said to be a method of sorting out common factors (i.e., principal components) from the five numerical values.

In FIG. 5, when the five variables are observed at their positions on coordinates, it may be seen that variables v1 to v3 and variables v4 to v5 belong to two respective clusters different from each other, and it may be said that the five variables are describable by using these two factors.

As a result, although the five variables are used as input values, it may be said that the variables used to predict actual biological age BA are factor 1 and factor 2.

Here, only one factor with the greatest influence is used in the actual biological age prediction model.

Unlike the multivariable linear regression (MLR) model, the biological age prediction model using PCA does not use chronological age CA as a dependent variable, but causes the extracted factors, expressing the greatest influence, to have the same measure (i.e., a unit) such as age (e.g., one year old, two years old), and causes the chronological age CA to be input into the biological age (BA) prediction model as an independent variable in order to correct bias in the biological age BA prediction.

In short, the PCA model can be expressed as Equation 2 below.

BA=F(X1)+G(CA) [Equation 2]
where, BA denotes biological age, X1 denotes one principal component factor extracted through PCA, CA denotes chronological age, F denotes conversion function using X1 as input variable, and G denotes conversion function using CA as input variable.

That is, the biological age means a numerical value calculated by multiplying the PCA principal component factors and the chronological age by respective weights, and then adding the multiplied results together.

### Weak Points of PCA Model

Since the principal components extracted through PCA have a very high correlation with the chronological age, an opinion that the calculated value is a numerical value representing a biological age is nothing but a researcher's subjective opinion.

In addition, since the opinion introduces a conversion function using “chronological age” as a parameter in order to make the factors extracted through PCA into the variable (i.e., biological age) having the unit of “age”, the opinion is not objectively proven and nothing but a simple idea of the researcher.

A yet another reason why “chronological age” is used as a parameter and included in the biological age model is because a phenomenon in which biological age is overestimated in a younger group and biological age is underestimated in an older group occurs in the same way as in the MLR model before using the “chronological age” as the parameter.

A method of calculating biological age by using the PCA biological age prediction model is provided in Korean Patent No. 0126229, in 2014, titled as “BIOLOGICAL AGE CALCULATION MODEL GENERATION METHOD AND SYSTEM THEREOF, BIOLOGICAL AGE CALCULATION METHOD AND SYSTEM THEREOF”.

### DISCLOSURE

### Technical Problem

In the Korean domestic environment where aging is rapidly progressing, a method for predicting aging status for each individual is required as a preventive measure to lead a healthier life of each individual for a long time.

An objective of the embodiment of the present disclosure is to provide a method and a system thereof for generating a personalized biological age prediction model that builds the biological age prediction model by gender and chronological age group in consideration of aging mechanisms that are different from each other depending on the gender and the chronological age group, and also enables predicting of biological age according to the biological age prediction model for each age group.

Another objective of the present disclosure is to provide a model and a service system thereof for predicting a personalized biological age that expresses an individual's aging status in a form of a biological age probability spectrum/distribution, rather than simply presenting only one numerical value of biological age (e.g., 55 years old), so that more objective and clear interpretations of biological age information may be provided.

### Technical Solution

In a biological age measurement model currently published in a form of papers or patents, only one numerical value is presented such that an individual's biological age is equal to 55.7 years. Since quantitative and qualitative interpretations of what this number means are not objective and are unclear, the individual's aging status is required to be expressed in a different form, such as a biological age probability spectrum/distribution, rather than a single numerical value.

The present disclosure is technically characterized in that unlike conventional biological age prediction models (i.e., MLR, PCA), “excessive aging factor (i.e., Δ)” unable to be described by chronological age is calculated through checkup data instead of directly predicting biological age by using the checkup data.

Since aging mechanisms are expected to be different from each other depending on gender or chronological age group, the present disclosure intends to develop a plurality of biological age measurement models that operate differently according to the gender and chronological age group.

The present disclosure is intended to predict biological age with a statistical model taking into account distribution of differences between checkup numerical values measured in individuals and values in comparison (e.g., average body mass index, average blood pressure, etc.) representing people of the same chronological age.

According to the present disclosure, a method for generating a personalized biological age prediction model is configured to include:
an age range setting process of setting an age range x to y to be used as training data in order to generate binary logistic regression models;
a binary logistic regression model generation process of setting each age unit as one unit in the age range set in the age range setting process, dividing the training data into two groups of an underage group UAGm and an overage group OAGm for each age unit, and generating the binary logistic regression models Mx to My for respective age units;
an age prediction probability calculation process of calculating a probability Pm to be predicted as the overage group OAGm for each individual, who is a sample target, according to the binary logistic regression models;
a cutoff extraction process of setting the underage group UAGm and the overage group OAGm as two-part response variables, setting the probability Pm to be predicted as the overage group OAGm as a predictor variable, and extracting a cutoff Cm through Receiver Operating Characteristic (ROC) curve analysis;
an age prediction probability correction process of calculating an excess probability Dm to be predicted as the overage group OAGm by applying (Pm−Cm) calculation to subtract the cutoff Cm from the probability Pm to be predicted as the overage group OAGm;
an excess age calculation process of obtaining an individual's excess age by obtaining a weighted mean Δi for every excess probability Dm to be predicted as the overage group OAGm obtained through the age prediction probability correction process; and
a biological age calculation process of obtaining a biological age by adding the individual's excess age obtained through the excess age calculation process to a chronological age.

In addition, the method may further include a checkup item information setting process of retrieving and setting to add or delete the checkup item information used as the training data, wherein the training data in the binary logistic regression model generation process may be organized according to the checkup item information.

In addition, the method may further include a condition information setting process of setting condition information for the training data in the binary logistic regression model generation process.

In the excess age calculation process,
the individual's excess age may be calculated by using a mean of a sum of each value obtained by multiplying the excess probability Dm (where, m=26, . . . , 75) calculated for each individual by corresponding age (=m).

According to the present disclosure, a system for generating a personalized biological age prediction model is configured to include:
a checkup data collection means configured to collect medical checkup data provided from a medical checkup system, and store and manage the medical checkup data in a data storage means;
a training data setting means configured to determine valid training data from the checkup data provided from the checkup data collection means according to a set training data reference age range x to y and checkup item information;
a binary logistic regression model generation means configured to generate binary logistic regression models Mx to My for respective age units within the age range x to y set for the training data set by the training data setting means;
an age prediction probability calculation means configured to calculate a probability Pm to be predicted as an overage group OAGm for each individual in the training data according to the binary logistic regression models generated by the binary logistic regression model generation means;
a cutoff extraction means configured to set an underage group UAGm and the overage group as two-part response variables, set the probability Pm to be predicted as the over-age group OAGm as a predictor variable, and extract a cutoff Cm through ROC curve analysis;
an age prediction probability correction means configured to apply (Pm−Cm) calculation to subtract the cutoff Cm from the probability Pm, which is to be predicted as the overage group OAGm and calculated through the age prediction probability calculation means, calculate an excess probability Dm to be predicted as the individual overage group OAGm, and correct the probability Pm, which is to be predicted as the overage group OAGm and calculated by the age prediction probability calculation means;
an excess age calculation means configured to obtain an individual's excess age by obtaining a weighted mean Δi for every excess probability Dm to be predicted as the overage group OAGm obtained through the age prediction probability correction means;
a biological age calculation means configured to calculate a biological age from a chronological age by using the individual's excess age obtained through the excess age calculation means; and
the data storage means configured to store and manage the medical checkup data collected from the checkup data collection means and the training data set through the training data setting means.

The system may further include a user setting means configured to provide a process enabling a user to retrieve and set the age range and the checkup item information of the training data setting means.

The system may further include a user setting means configured to provide a process enabling the user to set condition information for determining the training data in the training data setting means.

The checkup item information of the training data setting means may be composed of health insurance checkup item data including:
physical examination indices such as body mass index, waist circumference, systolic blood pressure, and diastolic blood pressure; and blood test indices such as three types of liver levels (i.e., AST, ALT, and γ-GTP), creatinine, three types of cholesterol (i.e., HDL, LDL, and TG), fasting blood glucose, and hemoglobin.

### Advantageous Effects

As described above, the embodiment of the present disclosure develops a biological age prediction model by using high-quality, large-scale medical checkup data already accumulated by the National Health Insurance Service, whereby the cost and time required in a process of separately creating and researching data for developing the biological age prediction model may be reduced.

In addition, the embodiment of the present disclosure enables each individual to use checkup data in consideration of the different degrees of aging according to gender and age group, calculates the individual's excess age by using relative values of each individual according to gender and age group, and predicts a biological age by using the individual's excess age as weight information, whereby a more reliable personalized biological age prediction model may be generated.

### DESCRIPTION OF DRAWINGS

FIG. 1 is a view illustrating an example of data distribution representing a correlation between chronological age and systolic blood pressure.

FIG. 2 is a view illustrating an example of data distribution representing a correlation between chronological age and hemoglobin.

FIG. 3 is a view illustrating a linear regression line in a multivariable linear regression (MLR) analysis model.

FIG. 4 is a graph illustrating a relationship between chronological age X and biological age Y.

FIG. 5 is a view illustrating a biological age prediction model using Principal Component Analysis (PCA).

FIG. 6 is a flowchart illustrating processes of a method for generating the personalized biological age prediction model according to the present disclosure.

FIG. 7 is a view illustrating a process of generating a binary logistic regression model in the present disclosure.

FIG. 8 is a table illustrating each probability value Pm obtained according to the binary logistic regression model in the present disclosure.

FIG. 9 is a table illustrating each cutoff value extracted through a cutoff extraction process in the present disclosure.

FIG. 10 is a table illustrating each excess probability Dm to be predicted as an overage group OAGm obtained through an age prediction probability correction process in the present disclosure.

FIG. 11 is a view illustrating an example of an excess age profile for each individual in the present disclosure.

FIG. 12 is a flowchart representing an exemplary embodiment for processes of generating a model to predict biological age in the present disclosure.

FIG. 13 is a block diagram illustrating a configuration of a system for generating a personalized biological age model of the present disclosure as described above.

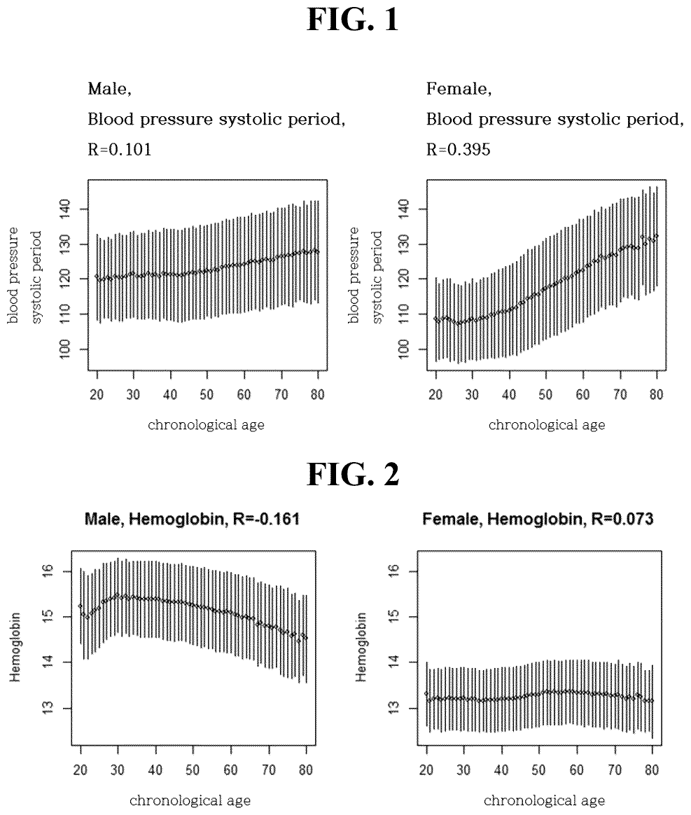

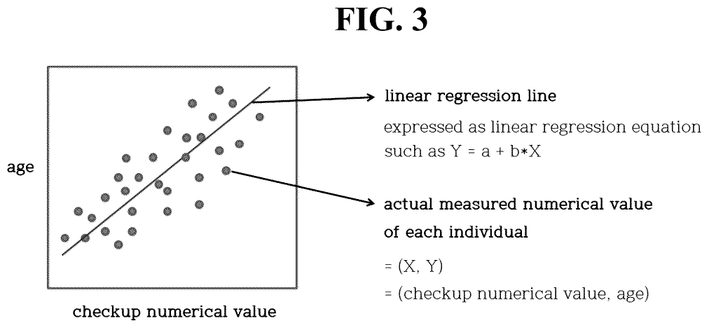

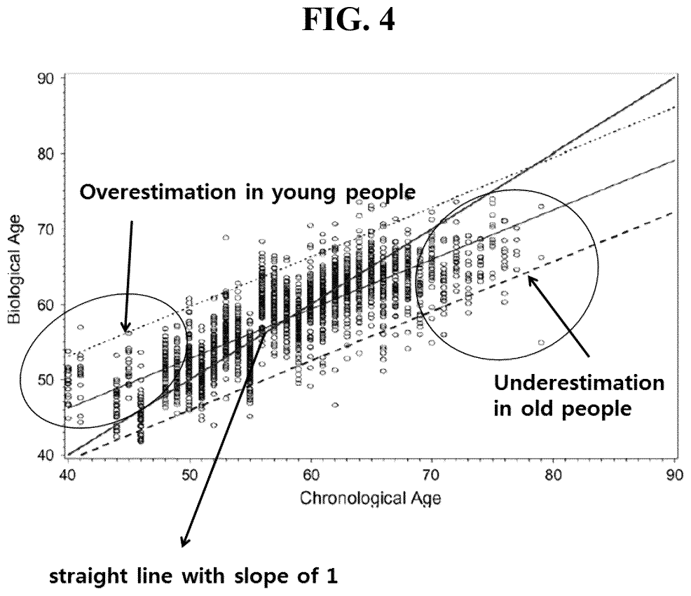

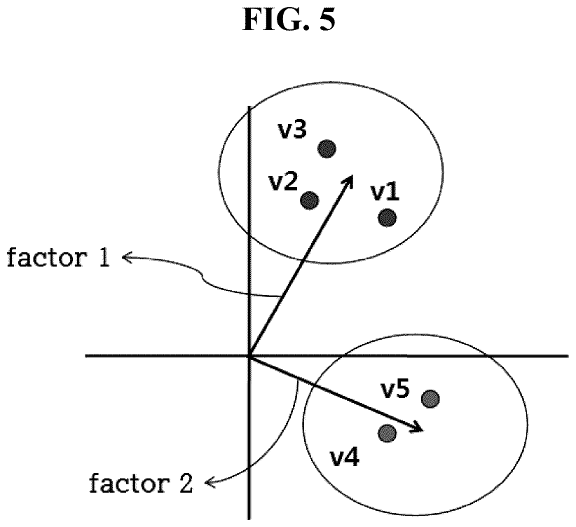

### BEST MODE

A method for generating a personalized biological age prediction model of the present disclosure is technically characterized by calculating an “excess aging factor (Δ)” that may not be described by chronological age through checkup data, and enabling a user to predict a biological age by using the excess aging factor.

The processes of generating the personalized biological age model of the present disclosure are configured as follows:
an age range setting process of setting an age range x to y to be used as training data in order to generate a binary logistic regression model; a binary logistic regression model generation process of setting each age unit as one unit in the age range that is set in the age range setting process, dividing the training data into two groups of an underage group UAGm and an overage group OAGm for each age unit, and generating binary logistic regression models Mx to My for respective age units;
an age prediction probability calculation process of calculating a probability Pm to be predicted as an overage group OAGm for each individual who is a sample target according to a binary logistic regression model;
a cutoff extraction process of setting the underage group UAGm and the overage group OAGm as two-part response variables, setting the probability Pm to be predicted as the overage group OAGm as a predictor variable, and extracting a cutoff Cm through ROC curve analysis;
an age prediction probability correction process of applying (Pm−Cm) calculation to subtract the cutoff Cm from the probability Pm to be predicted as the overage group OAGm, and calculating an excess probability Dm to be predicted as the overage group OAGm;
an excess age calculation process of obtaining a weighted mean Δi for every excess probability Dm to be predicted as the overage group OAGm obtained through the age prediction probability correction process, and obtaining an individual's excess age; and
a biological age calculation process of obtaining a biological age by adding the individual's excess age obtained through the excess age calculation process to a chronological age.

The biological age prediction model of the present disclosure may be defined as multivariable binary logistic regression (MBLR) analysis, and its characteristics may be simplified as follows.

Biological age prediction model (using MBLR) of the present disclosure;

Biological age BA=chronological age CA+Δ

Δ=f(BMI, SBP, . . . , CA)
where, f(BMI, SBP, . . . ) represents over-aging factor calculation function based on binary logistic regression model using medical checkup numerical values as input variables.

In contrast to this, the conventional MLR model and PCA model may be expressed as follows.

MLR model: BA=a0+a1×BMI+a2×SBP+ . . .

PCA model: BA=F(BMI, SBP, . . . )+G(CA)

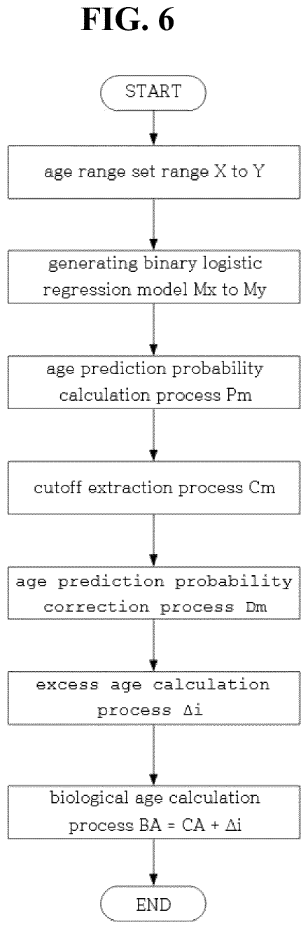

In the present disclosure configured as described above,
as shown in FIG. 6, the method of the present disclosure is technically characterized by enabling a user to obtain an excess age Δi for a chronological age CA in obtaining a biological age BA, and is configured to include:
(a) an age range setting process,
(b) a binary logistic regression model generation process,
(c) an age prediction probability calculation process,
(d) a cutoff extraction process,
(e) an age prediction probability correction process,
(f) an excess age calculation process,
(g) a biological age calculation process.

In the age range setting process,
this process is for setting targets of health insurance checkup data in order to use training data for obtaining biological age, and setting an age range x to y used to obtain a binary logistic regression model.

The exemplary embodiment of the present disclosure sets 26 (i.e., x) to 75 (i.e., y) years of age as the targets of the health insurance checkup data.

The above 26 and 75 years old are values used due to the characteristics of health insurance data, and in a case of using data other than the health insurance data, x (i.e., 26 years old) and y (i.e., 75 years old) may be changed.

The binary logistic regression model generation process is a process of generating a binary logistic regression model for obtaining a probability Pm to be predicted as an overage OAGm in two groups, and is a process of generating a model that may divide “chronological age” into the two groups and predicting any one group OAGm of these two groups.

Age units that may be set in the range of 26 to 75 years old that is set above are 50 units. For each unit, training data for each checkup item is divided into two groups: an underage group UAGm and an overage group OAGm.

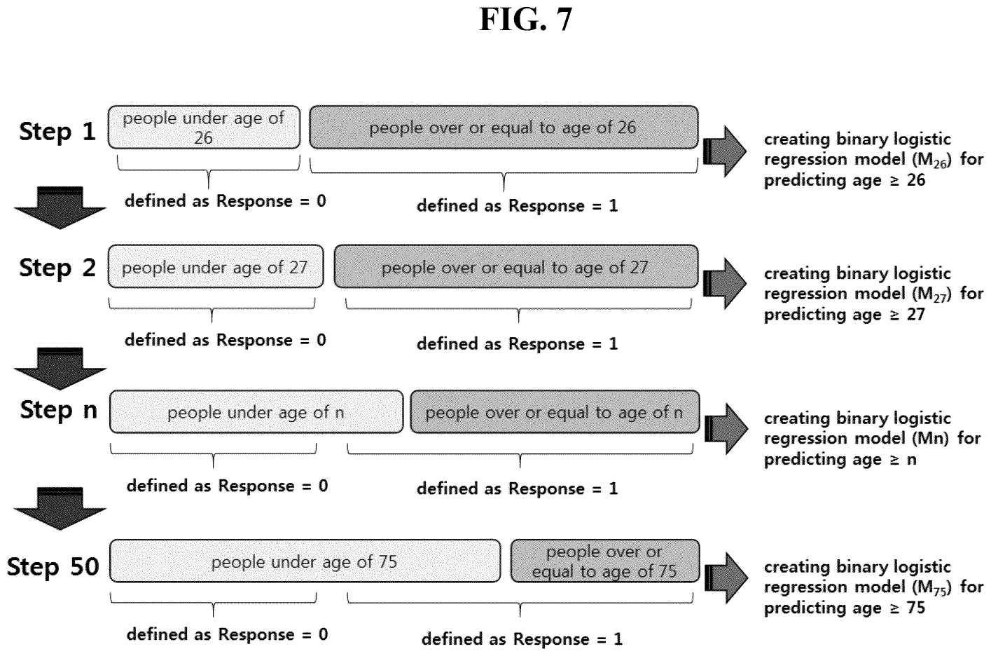

FIG. 7 is a view illustrating a process of generating a binary logistic regression model.

As shown in FIG. 7, in each unit age, age is divided into a group of age under a corresponding age UAGm and a group of age over or equal to a corresponding age OAGm, any one of the two groups is selected as training data in each unit, and the total of 50 binary logistic regression models are generated.

For example, a group of age under the age of 26 and a group of age over or equal to the age of 26 are set in the 26-year-old unit, and the group of age under the age of 26 is classified into 0 and the group of age over or equal to the age of 26 is classified into 1 in a unit of checkup item data, which is set as training data, so that a binary logistic regression model M26 is generated to predict the age of 26 or older in the age prediction probability calculation process. For specific values for each checkup item, people under the age of 26 are classified as “0” and those over or equal to the age of 26 are classified as “1”, thereby generating the binary logistic regression model M26.

The binary logistic regression model M26 is generated by dividing people into people under the age of 26 and those with values over or equal to the age of 26 for each piece of checkup data of health insurance checkup items, including: physical examination indices such as body mass index, waist circumference, systolic blood pressure, and diastolic blood pressure; and blood test indices such as three type of liver levels (i.e., AST, ALT, and γ-GTP), creatinine, three types of cholesterol (i.e., HDL, LDL, and TG), fasting blood glucose, and hemoglobin.

That is, a binary logistic regression model is generated according to two groups, i.e., an underage group UAGm and an overage group OAGm, as response variables on a Y-axis, and training data (i.e., checkup data) as a predictor variable on an X-axis.

A checkup item information setting process may be further included, so that the above health insurance checkup items to be used as the training data are to be retrieved, and are set to be added or deleted by using checkup item information.

In addition, a condition information setting process of setting condition information for the training data may be further included, and the condition information may be organized with male and female gender information.

According to this configuration, biological age prediction models according to male and female gender may be generated separately.

The total of 50 binary logistic regression models M26 to M75 is generated by performing such processes for years of age from 26 to 76, iteratively.

The age prediction probability calculation process is a process of calculating a probability Pm to be predicted as an overage group OAGm for each individual according to the binary logistic regression models M26 to M75 generated as described above.

Equation 3 below shows the age prediction probability calculation process according to the binary logistic regression models.

$$p ( Y = {OAG}_{m} ) = \frac{exp ( {\sum }_{k = 0}^{p} {β}_{k} {X}_{k} )}{1 + exp ( {\sum }_{k = 0}^{p} {β}_{k} {X}_{k} )} \qquad [ Equation 3 ]$$

$$Define {P}_{im} = p ( {Y}_{i} = {OAG}_{m} )$$

where,
Y: individual's aging status
p(Y=OAGm): probability to be predicted as overage group OAGm
Yi: i-th individual's aging status
i=1, 2, . . . , : sample number
m=26 (i.e., x), 27, . . . , 75 (i.e., y): age used for training data
(chronological age observed in the training data)
CA: chronological age
Xk: k-th independent variable
βk: regression coefficient of k-th independent variable
p: number of independent variables

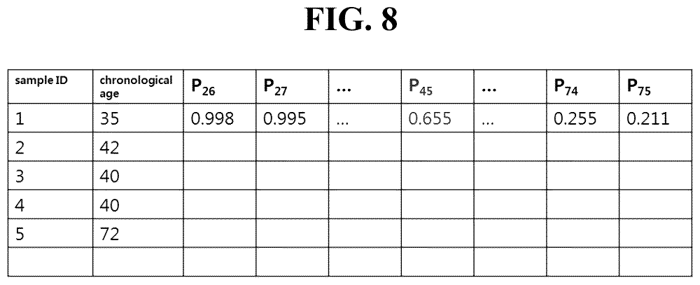

FIG. 8 is a table illustrating each probability value Pm obtained according to the binary logistic regression model.

In the table of FIG. 8, a probability value “P45”, which is the probability value obtained by using a binary logistic regression model M45, means the probability value predicted to be 45 years old or older.

For example, for a person with sample ID=1, the probability P45 to be predicted as 45 years old or older is 0.655, and a probability to be predicted as 75 years old or older is 0.211.

The age prediction probability calculation process calculates these 50 probability values (i.e., P26 to P75) for each person (each sample) for all ages.

That is, the probability values Pm are obtained for each individual for all age units.

Here, as shown in FIG. 8, when a probability P26 to be predicted as an overage group OAG26 is examined, it may be seen that the probability P26 is 0.998, which is close to 1.

This is inaccurate in a case where biological age is predicted with respect to each probability Pm as described above as an absolute value, so a relative value is required to be applied, whereby a more accurate biological age prediction is available.

Accordingly, cutoffs Cm, i.e., reference values for determining biological age, are required.

The cutoff extraction process is a process of obtaining a reference value for determining biological age through Receiver Operating Characteristic (ROC) curve analysis and Area Under the Curve analysis, which are performed on probability values Pm obtained for 50 models M26 to M75 for all people aged 26 to 75, and is a process of setting the underage group UAGm and the overage group OAGm as two-part response variables, setting the probability Pm to be predicted as the overage group OAGm as a predictor variable, and extracting a cutoff Cm through the ROC curve analysis.

Such a cutoff extraction process extracts the cutoff Cm at a time point of maximizing Youden's J statistic, resulting in extracting of the cutoff at which the sum of sensitivity and specificity is maximized.

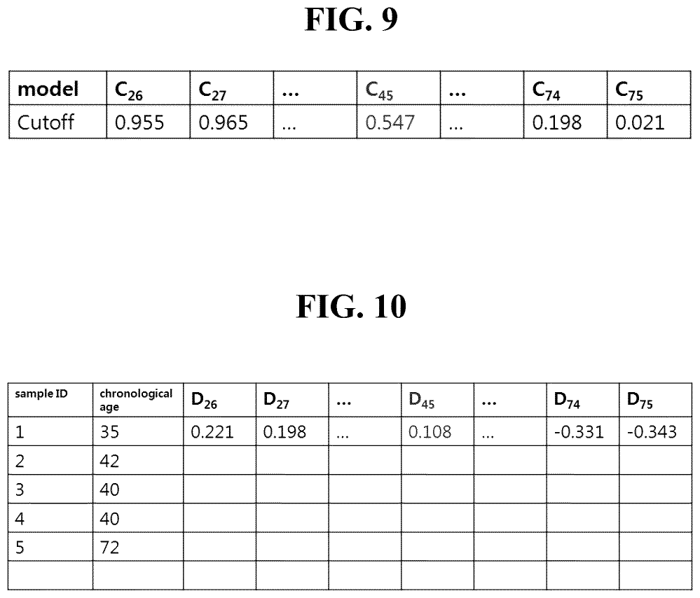

FIG. 9 is a table illustrating each cutoff value extracted through the cutoff extraction process.

For example, in the table of FIG. 9, C45 is a cutoff value obtained from a model M45, and when a probability value is calculated as 0.547 or higher, the probability means that age of a corresponding person is predicted to belong to a group of age over or equal to the age of 45 years old.

The age prediction probability correction process is a process of correcting a probability Pm to an excess probability Dm to be predicted as an overage group OAGm by applying (Pm−Cm) calculation to subtract a cutoff value Cm obtained through the age prediction probability calculation process from the probability Pm to be predicted as the overage group OAGm.

FIG. 10 is a table illustrating each excess probability Dm to be predicted as the overage group OAGm obtained through the age prediction probability correction process.

In the table of FIGS. 10, D26 to D75 are values obtained by respectively subtracting cutoffs C26 to C75 calculated through the ROC curve from 50 probability values P26 to P75 calculated for each individual. (i.e., Dm=Pm−Cm)

For example, when a chronological age of a person with ID=1 is 35 years old, D45 that is a probability of this person to be predicted as 45 years old or older is equal to “D45=0.108 (P45−C45, 0.655−0.547)”.

Here, in a case of a (−) value, this value may be considered as a value of age less than the corresponding age.

The excess age calculation process is a process of obtaining a weighted mean Δi for every excess probability Dm to be predicted as the overage group OAGm obtained through the above process, and obtaining an individual's excess age to obtain his or her biological age.

Equation 4 below shows a process of calculating a weighted mean Δi for every excess probability Dm to be predicted as an overage group OAGm.

$${Δ}_{i} = \frac{{\sum }_{m = x}^{y} {m}^{⋆} ( {P}_{im} - {C}_{m} )}{y - x + 1} \qquad [ Equation 4 ]$$

where, N: sample number i=1, 2, . . . , N
Δi: weighted mean of (Pim−Cm)
Cm: value of cutoff Cm obtained through cutoff extraction means 150
(cutoff of Pm to predict individual's aging status from ROC curve analysis)

That is, a mean of the sum of each value obtained by multiplying Dm (where, m=26, . . . , 75) calculated for each individual by corresponding age (=m) is defined as an “excess age” of each individual.

Here, the individual's excess age is obtained by the weighted mean for every excess probability Dm predicted as the overage group OAGm, and accordingly, in a case where there is an additional weight Wm to be applied, a weighted mean may be obtained by applying the additional weight Wm.

Equation 5 below shows a process of calculating a weighted mean Δi for every excess probability Dm to be predicted as an overage group OAGm.

$${Δ}_{i} = \frac{{\sum }_{m = x}^{y} {{m}^{⋆} ( {P}_{im} - {C}_{m} )}^{⋆} {w}_{m}}{y - x + 1} \qquad [ Equation 5 ]$$

$${\sum }_{x}^{y} {w}_{m} = 1$$

where, N: sample number i=1, 2, . . . , N
Δi: weighted mean of (Pim−Cm)
Cm: value of cutoff Cm obtained through cutoff extraction means 150
(cutoff of Pm to predict individual's aging status from ROC curve analysis)
Wm: weight applied for model to predict chronological age, CA≥m

The biological age calculation process is a process of obtaining a biological age by adding the excess age obtained in the excess age calculation process to chronological age.

As such, the present disclosure is technically characterized by generating a model (i.e., an algorithm) for predicting biological age by using health insurance checkup data.

In the present disclosure, biological age may be predicted by obtaining excess age Ai for chronological age CA.

First, a target of health insurance checkup data is set in order to use training data for obtaining biological age.

In the exemplary embodiment of the present disclosure, age of 26 to 75 years old is set as the training data age target x to y, which is an age range for obtaining binary logistic regression models.

As described above, in consideration of the characteristics of the health insurance checkup data, 26 to 75 years old is set as the age range for obtaining the binary logistic regression models.

In addition, a checkup item information setting process of setting, as checkup item information, checkup items to be used as the training data may be further included, and a user (or an administrator) may set the checkup items to be used as the training data in order to predict biological age.

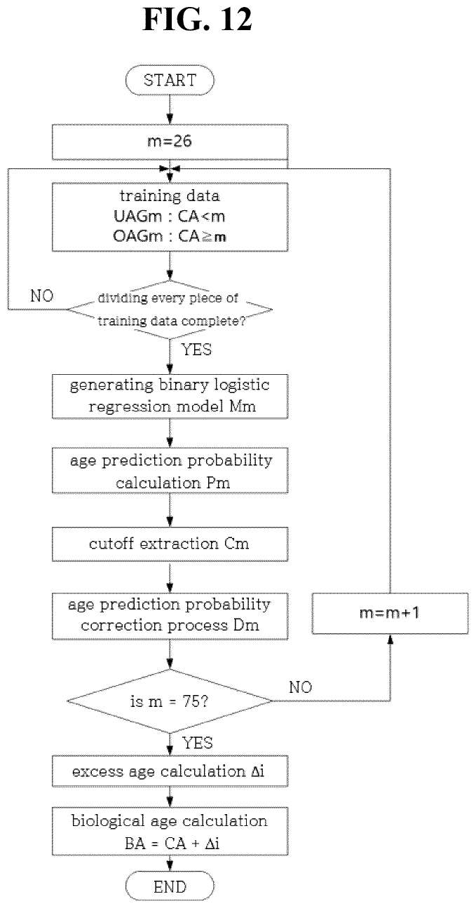

FIG. 12 is a flowchart representing the exemplary embodiment for processes of generating a model to predict biological age in the present disclosure. Referring to FIG. 12, the exemplary embodiment of the operation process will be described as follows.

First, age to be used for training data is initialized, and m=26 years old is set.

After that, according to the training data, age is divided into an underage group UAG26 under the age of 26 and an overage group OAG26 over or equal to the age of 26.

That is, medical checkup data is divided into a group of age under the age of 26 and a group of age over or equal to the age of 26. A sample target (i.e., a person) of the checkup data is checked for specific values for each medical checkup item, the sample (the person) under the age of 26 is set as an underage group UAGm “0”, and the sample (person) over or equal to the age of 26 is set as an overage group OAGm “1”, thereby generating a binary logistic regression model M26 corresponding to the age of 26.

The binary logistic regression model is for obtaining a probability Pm to be seen as an overage group OAGm in two groups. As described above, the binary logistic regression model uses each piece of checkup data of health insurance checkup items, including: physical examination indices such as body mass index, waist circumference, systolic blood pressure, and diastolic blood pressure; and blood test indices such as three types of liver levels (i.e., AST, ALT, and γ-GTP), creatinine, three types of cholesterol (i.e., HDL, LDL, and TG), fasting blood glucose, and hemoglobin. When necessary, these checkup items may be added or deleted to be set as checkup item information.

Thereafter, according to the binary logistic regression model M26 generated as described above, a probability P26 to be predicted as an overage group OAG26 for each individual is calculated through Equation 3 above to obtain an age prediction probability.

That is, such an age prediction probability represents individual's aging status, and represents a probability to be predicted as an overage group OAGm.

Thereafter, as described above, a cutoff Cm, which is a reference value for determining biological age, is obtained. This process is performed by setting the underage group UAGm and the overage group OAGm as two-part response variables, setting the probability Pm to be predicted as the overage group OAGm as a predictor variable, and extracting the cutoff Cm through ROC curve analysis, whereby a cutoff value C26 for determining a biological age is obtained through the ROC curve analysis for the probability P26 to be predicted as 26 years old or older.

Thereafter, a process of correcting the age prediction probability is performed by applying the cutoff C26 obtained as described above.

In the age prediction probability correction process, an excess probability D26 to be predicted as an overage group OAG26 is obtained by performing (P26−C26) calculation to subtract the cutoff value C26 obtained through the age prediction probability calculation process from the probability P26 to be predicted as the overage group OAG26.

As such, the excess probability D26 to be predicted as the overage group OAG26 is obtained by applying the cutoff C26 for each individual.

As described above, when every excess probability up to the excess probability D26 to be predicted as the overage group OAG26 for each individual (i.e., each sample) is obtained, a current process is returned to an initial process to set m=27, and through the processes described above, a binary logistic model M27, a probability P27 to be predicted as an overage group OAG27, a cutoff C27, and an excess probability D27 to be predicted as the overage group OAG27 are obtained.

By iteratively performing such a process until m=75, an excess probability D75 to be predicted as an overage group is obtained for each individual.

For each age unit, training data is divided into an underage group UAGm and an overage group OAGm, and as shown in FIG. 7, respective binary logistic regression models are generated for 50 models.

As described above for example, the binary logistic regression model M26 is generated by dividing people with a value of age under 26 years old and those with a value of age over or equal to 26 years old for the training data including: physical examination indices such as body mass index, waist circumference, systolic blood pressure, and diastolic blood pressure; and blood test indices such as three types of liver levels (i.e., AST, ALT, and γ-GTP), creatinine, three types of cholesterol (i.e., HDL, LDL, and TG), fasting blood glucose, and hemoglobin. Binary logistic regression models M27 to M75 are generated By iteratively performing these processes for the ages of 27, 28, . . . , 75.

As shown in FIG. 8 that illustrates individual's aging status according to the binary logistic regression models M26 to M75 generated as described above, every probability Pm to be predicted as the overage group OAGm is obtained by calculating each Pm (i.e., P26 to P75) in all age units (m=26 to 75).

As shown in the examples described above, this means that a probability P45 that a person with sample ID=1 belongs to a group of age over or equal to age of 45 years old is 0.655, and a probability that the person belongs to a group of age over or equal to age of 75 years old is 0.211.

The cutoffs C26 to C75 obtained as above are values extracted through the ROC curve analysis, and this mean that each cutoff Cm is extracted at a time point of maximizing Youden's J statistic.

An excess probability Dm to be predicted as an overage group OAGm calculated through the age prediction probability correction process is obtained by applying the cutoff Cm to the probability Pm to be predicted as the overage group OAGm obtained in the age prediction probability process, and as shown in FIGS. 10, D26 to D75 for the respective age from 26 to 75 years old are obtained for each individual.

When this process is iteratively performed up to m=75 so that every excess probabilities up to an excess probability D75 to be predicted as an overage group OAG75 is obtained for each individual, a weighted mean Δi is obtained for every excess probability Dm to be predicted as the overage group OAGm obtained through the above processes, so that an individual's excess age is obtained to obtain his or her biological age.

Through Equation 4 above, the individual's excess age as above may be obtained by using the weighted mean Δi.

That is, according to Equation 4, a mean of the sum of each value obtained by multiplying the Dm calculated for each individual (m=26, . . . , 75) by the corresponding age (=m) is defined as an “excess age” of each individual.

A biological age may be obtained by applying, to a chronological age, the weighted mean obtained in this way as the individual's excess age.

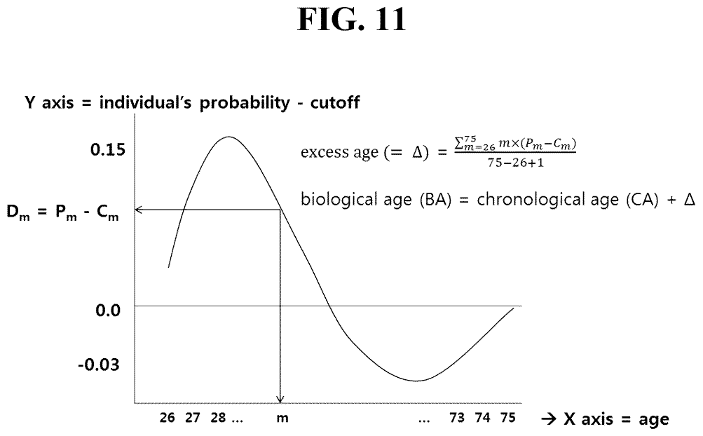

FIG. 11 is a view illustrating an example of an excess age profile for each individual. The X-axis is set to be training data age targets 26 to 75, and the Y-axis is set to be excess probability Dm to be predicted as overage group OAGm, so as to represent the excess probability Dm to be predicted as the overage group OAGm for each age target.

As described above, the embodiment of present disclosure obtains average information of information representing each individual's aging status by using the health insurance checkup data, and accordingly generates the model (i.e., the algorithm) capable of predicting biological age.

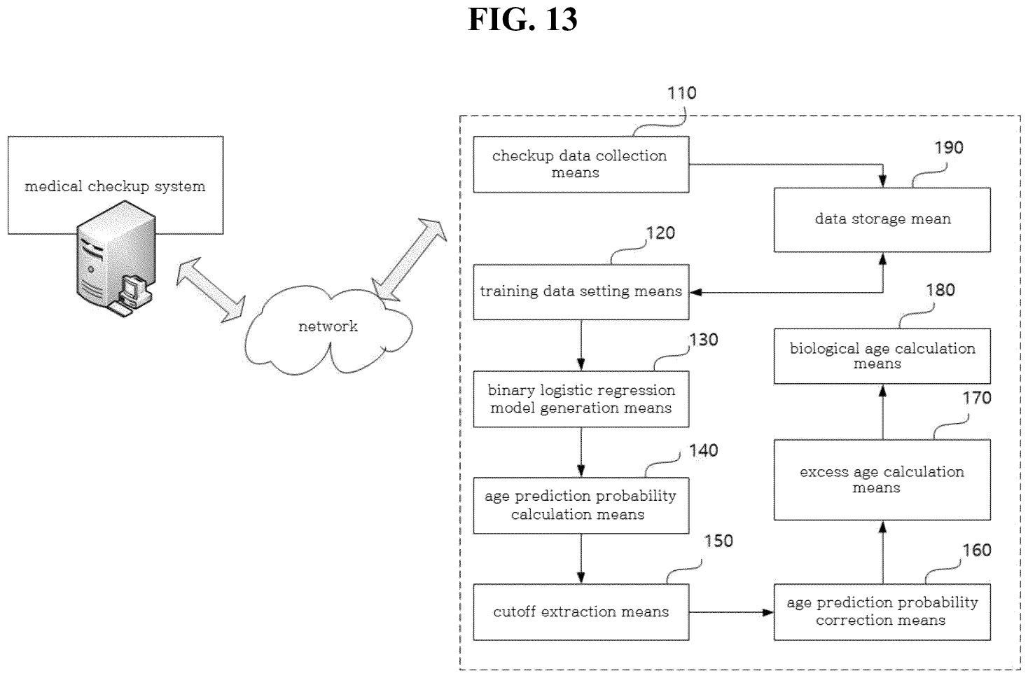

Meanwhile, FIG. 13 is a view illustrating a configuration of a system for generating a personalized biological age model according to the present disclosure as described above.

The system includes: a checkup data collection means 110 configured to collect medical checkup data provided from a medical checkup system, and store and manage the medical checkup data in a data storage means 190;
a training data setting means 120 configured to determine valid training data from the checkup data collected from the checkup data collection means 110 according to a set training data reference age range x to y and checkup item information;
a binary logistic regression model generation means 130 configured to generate binary logistic regression models Mx to My for respective age units within the age range x to y set for the training data set by the training data setting means 120;
an age prediction probability calculation means 140 configured to calculate a probability Pm to be predicted as an overage group OAGm for each individual in the training data according to a binary logistic regression model generated by the binary logistic regression model generation means 130;
a cutoff extraction means 150 configured to set an underage group UAGm and the overage group OAGm as two-part response variables, set the probability Pm to be predicted as the overage group OAGm as a predictor variable, and extract a cutoff Cm through ROC curve analysis;
an age prediction probability correction means 160 configured to apply (Pm−Cm) calculation to subtract the cutoff Cm from the probability Pm, which is to be predicted as the overage group OAGm and calculated through the age prediction probability calculation means 140, calculate an excess probability Dm to be predicted as the individual overage group OAGm, and correct the probability Pm, which is to be predicted as the overage group OAGm and calculated by the age prediction probability calculation means 140;
an excess age calculation means 170 configured to calculate an individual's excess age by obtaining a weighted mean Ai for every excess probability Dm to be predicted as the overage group OAGm obtained through the age prediction probability correction means 160;
a biological age calculation means 180 configured to calculate a biological age from a chronological age by using the individual's excess age obtained through the excess age calculation means 170; and
a data storage means 190 configured to store and manage the medical checkup data collected from the checkup data collection means 110 and the training data, which is set through the training data setting means 120.

As such, the personalized biological age prediction system of the present disclosure is technically characterized by setting the training data from the medical checkup data provided from the medical checkup system, and extracting individual's excess age information therefrom, and predicting his or her biological age.

A biological age prediction model generation system is configured to receive medical checkup data from a medical checkup system, and generate personalized biological age models.

In the above biological age prediction model generation system,
the checkup data collection means 110 is a means configured to collect the medical checkup data provided from the medical checkup system, and is a means configured to store and manage the collected medical checkup data in the data storage means 190.

The training data setting means 120 is a means configured to set the training data for generating biological age prediction models, and is a means configured to determine valid training data of the binary logistic regression model generation means from the checkup data stored in the data storage means 190 according to the set training data reference age range x to y and the checkup item information.

The binary logistic regression model generation means 130 is a means configured to generate binary logistic regression models Mx to My for respective age units within the age range set for the training data set by the training data setting means 120.

The binary logistic regression model generation means 130 is a means configured to set each age unit as one unit in the set age range, divide the training data for each age unit into two groups of an underage group UAGm and an overage group OAGm, set the training data (i.e., checkup data) and the two groups of underage group UAGm and overage group OAGm as response variables, and generate the binary logistic regression models Mx to My for the respective age units.

The age prediction probability calculation means 140 is a means configured to calculate the probability Pm to be predicted as the overage group OAGm for each individual according to 50 binary logistic regression models generated by the binary logistic regression model generation means 130.

The cutoff extracting means 150 is a means configured to extract a cutoff Cm for correcting the probability Pm to be predicted as the overage group OAGm calculated through the age prediction probability calculation means 140, and is a means configured to set the underage group UAGm and the overage group OAGm as the two-part response variables, set the probability Pm to be predicted as the overage group OAGm as a predictor variable, and extract the cutoff Cm through the ROC curve analysis.

The age prediction probability correction means 160 is a means configured to correct the probability Pm to be predicted as the overage group OAGm calculated through the age prediction probability calculation means 140, and is a means configured to apply (Pm−Cm) calculation to subtract the cutoff Cm from the probability Pm to be predicted as the overage group OAGm, calculate an excess probability Dm to be predicted as the individual overage group OAGm, and correct the probability Pm to be predicted as the overage group OAGm calculated by the age prediction probability calculation means 140.

The excess age calculation means 170 is a means configured to obtain the individual's excess age for obtaining a biological age, and is a means configured to obtain a weighted mean Δi for every excess probability Dm to be predicted as the overage group OAGm obtained through the age prediction probability correction means 160, and obtain the individual's excess age.

The biological age calculation means 180 is a means configured to calculate the biological age from a chronological age by using the individual's excess age obtained through the excess age calculation means 170.

Operations of the system of the present disclosure having such a configuration will be described as follows.

The checkup data collection means 110 collects checkup data provided from a medical checkup system and stores the checkup data in a data storage means 190.

The training data setting unit 120 sets training data for obtaining binary logistic regression models from the medical checkup data stored in the data storage unit 190.

The training data setting unit 120 determines the training data for a set age range x to y and medical checkup items.

The exemplary embodiment of the present disclosure uses health insurance checkup data, and the age range is set to be a range from 26 years old as x to 75 years old as y.

The embodiment of the present disclosure may be configured to further include a user setting means configured to provide a process enabling a user (or an administrator) to retrieve and reset the age range and the checkup item information of the training data setting means 120.

In addition, the embodiment of the present disclosure may be configured to further include a user setting means configured to provide a process enabling the user to set condition information for determining training data in the training data setting means 120.

The condition information may be composed of male and female gender information, and may be organized by setting the male and female gender information and by dividing biological age prediction models according to male and female gender.

Thereafter, the binary logistic regression model generation means 130 sets 50 units for respective age units within the age range of the training data setting means 120, divides the training data for each unit into two groups of an underage group UAGm and an overage group OAGm, and generates binary logistic regression models.

This is a process of generating each binary logistic regression model for obtaining a probability Pm to be seen as an overage group OAGm in the two groups.

A binary logistic regression model M26 is generated by setting a group of age under the age of 26 UAG26 and a group of age over or equal to the age of 26 OAG26 in a unit of m=26 and by dividing samples (i.e., people) under the age of 26 as 0 and samples (people) over or equal to the age of 26 as 1 for each piece of training data.

That is, the binary logistic regression model M26 is generated by dividing people into people under the age of 26 and people over or equal to the age of 26 for the training data on such health insurance checkup items including: physical examination indices such as body mass index, waist circumference, systolic blood pressure, and diastolic blood pressure; and blood test indices such as three types of liver levels (i.e., AST, ALT, and γ-GTP), creatinine, three types of cholesterol (i.e., HDL, LDL, and TG), fasting blood glucose, and hemoglobin.

That is, each binary logistic regression model is generated by setting the two groups of the underage group UAGm and overage group OAGm as the response variables on the Y-axis, and setting the training data (i.e., the checkup data for each checkup item) as the predictor variable on the X-axis.

The total of 50 binary logistic regression models M26 to M75 are generated by iteratively performing such a process from 26 to 76 years of age.

When the binary logistic models are generated as described above, each probability Pm to be predicted as the overage group OAGm for each individual is calculated according to the binary logistic regression models M26 to M75 generated as described above.

As such, the probability Pm to be predicted as the overage group OAGm is information for obtaining an individual's excess age in order to predict his or her biological age, and may be obtained through Equation 3 above.

As shown in FIG. 8, the probability values Pm for each individual may be obtained according to the binary logistic regression models.

For example, it means that a person with sample ID=1 has a probability P45 to belong to a group of age over or equal to the age of 45 year old is 0.655 and a probability to belong to a group of age over or equal to the age of 75 years old is 0.211.

Meanwhile, the cutoff extraction means 150 extracts a cutoff Cm for the probability Pm to be predicted as the overage group OAGm for each individual through the ROC curve analysis.

The cutoff Cm is a reference value for determining a biological age, and such a value of each cutoff Cm in FIG. 9 may be obtained by setting the underage group UAGm and the overage group OAGm as two-part response variables, setting the probability Pm to be predicted as the overage group OAGm as the predictor variable, and performing the ROC curve analysis.

Thereafter, the age prediction probability correction means 160 corrects the probability Pm to be predicted as the overage group OAGm obtained by the age prediction probability calculation means 140 by using the cutoff value Cm obtained by the cutoff extraction means 150.

Such age prediction probability correction is to calculate an excess probability Dm to be predicted as the overage group OAGm by applying (Pm−Cm) calculation to subtract the cutoff value Cm obtained through the age prediction probability calculation means 140 from the probability Pm to be predicted as the overage group OAGm, and may obtain the excess probability Dm, which is to be predicted as the overage group OAGm and corrected for each individual as shown in FIG. 10.

According to FIG. 10, when a chronological age of the person with ID=1 is 35 years old and calculation is performed with a D45 model, that is, when a probability D45 that this person is predicted to belong to a group of age over or equal to the age of 45 years old is D45, the probability D45 is calculated as “D45=0.108 (P45−C45; 0.655−0.547)”.

Here, in a case of a (−) value, it may be considered as a value of age less than the corresponding age.

The excess age calculation means 170 obtains a weighted mean Δi through Equation 4 for every excess probability Dm to be predicted as the overage group OAGm, thereby obtaining an individual's excess age.

In this case, the individual's excess age is obtained by the weighted mean of every excess probability Dm to be predicted as the overage group OAGm, and in a case where there is an additional weight Wm to be applied, a weighted mean may be obtained by applying the additional weight Wm as in Equation 5 above.

The biological age calculation means obtains a biological age (BA=CA+Δi) from the chronological age by using the excess age obtained by the excess age calculation means.

According to the present disclosure as described above, the present disclosure calculates the excess age for the chronological age from the health insurance checkup data and enables the biological age to be predicted, whereby a more reliable biological age may be provided.

### INDUSTRIAL APPLICABILITY

The present disclosure has developed a biological age prediction model by using high-quality, large-scale medical checkup data accumulated by the National Health Insurance Corporation, and the present disclosure is a technology that may be widely used in the medical and statistical analysis industries and may realize practical and economical values.

1. A method for generating a personalized biological age prediction model, the method being performed in a system thereof for generating the personalized biological age prediction model to generate the biological age prediction model from medical checkup data collected from a medical checkup system, and the method comprising:
an age range setting process of setting, by a training data setting means (120), an age range (x to y) to be used as training data in order to generate binary logistic regression models;
a binary logistic regression model generation process of setting, by a binary logistic regression model generation means (130), each age unit as one unit in the age range set in the age range setting process, dividing the training data into two groups of an underage group (UAGm) and an overage group (OAGm) for each age unit, and generating the binary logistic regression models (Mx to My) for respective age units;
an age prediction probability calculation process of calculating, by an age prediction probability calculation means (140), a probability (Pm) to be predicted as the overage group (OAGm) for each individual, who is a sample target, according to the binary logistic regression models;
a cutoff extraction process of setting, by a cutoff extraction means (150), the underage group (UAGm) and the overage group (OAGm) as two-part response variables, setting the probability (Pm) to be predicted as the overage group (OAGm) as a predictor variable, and extracting a cutoff (Cm) through Receiver Operating Characteristic (ROC) curve analysis;
an age prediction probability correction process of calculating, by an age prediction probability correction means (160), an excess probability (Dm) to be predicted as the overage group (OAGm) by applying (Pm−Cm) calculation to subtract the cutoff (Cm) from the probability (Pm) to be predicted as the overage group (OAGm);
an excess age calculation process of obtaining, by an excess age calculation means (170), an individual's excess age by obtaining a weighted mean (Δi) for every excess probability (Dm) to be predicted as the overage group (OAGm) obtained through the age prediction probability correction process; and
a biological age calculation process of obtaining, by a biological age calculation means (180), a biological age by adding the individual's excess age obtained through the excess age calculation process to a chronological age.

2. The method of claim 1, wherein the training data in the binary logistic regression model generation process is organized according to checkup item information, and
the checkup item information is composed of health insurance checkup item data comprising:
physical examination indices such as body mass index, waist circumference, systolic blood pressure, and diastolic blood pressure; and
blood test indices such as three types of liver levels (i.e., AST, ALT, and γ-GTP), creatinine, three types of cholesterol (i.e., HDL, LDL, and TG), fasting blood glucose, and hemoglobin.

3. The method of claim 1 or 2, further comprising:
a checkup item information setting process of retrieving and setting to add or delete the checkup item information used as the training data,
wherein the training data in the binary logistic regression model generation process is organized according to the checkup item information.

4. The method of claim 1, further comprising:
a condition information setting process of setting condition information for the training data in the binary logistic regression model generation process.

5. The method of claim 4, wherein the condition information in the condition information setting process is male and female gender information.

6. The method of claim 1, wherein, in the binary logistic regression model generation process, the binary logistic regression models (Mx to My) are generated for the respective age units by setting each age unit as one unit in the set age range, dividing the training data for each age unit into the two groups of the underage group (UAGm) and the overage group (OAGm), setting the two groups of the underage group (UAGm) and the overage group (OAGm) as the response variables, and setting the training data as the predictor variable.

7. The method of claim 1, wherein, in the age prediction probability calculation process, calculating of the probability (Pm) to be predicted as the overage group (OAGm) for each individual, who is the sample target, according to the binary logistic regression model is calculated by Equation below:
$$p ( Y = {OAG}_{m} ) = \frac{exp ( {\sum }_{k = 0}^{p} {β}_{k} {X}_{k} )}{1 + exp ( {\sum }_{k = 0}^{p} {β}_{k} {X}_{k} )} ,$$
$$Define {P}_{im} = p ( {Y}_{i} = {OAG}_{m} )$$
where, Y: individual's aging status,
p(Y=OAGm): probability to be predicted as overage group OAGm,
Yi: i-th individual's aging status,
i=1, 2, . . . , : sample number,
m=26 (as x), 27, . . . , 75 (as y): chronological age observed in training data,
CA: chronological age,
Xk: k-th independent variable,
βk: regression coefficient of k-th independent variable, and
p: number of independent variables.

8. The method of claim 1, wherein, in the excess age 10 calculation process, the individual's excess age is calculated by Equation below, expressing a mean of a sum of each value obtained by multiplying the excess probability (Dm) (where, m=26, . . . , 75) calculated for each individual by corresponding age (=m):
$${Δ}_{i} = \frac{{\sum }_{m = x}^{y} {m}^{⋆} ( {P}_{im} - {C}_{m} )}{y - x + 1}$$
where, N: sample number i=1, 2, . . . , N,
Δi : weighted mean of (Pim−Cm), and
Cm: cutoff value Cm obtained through age prediction probability calculation process
(cutoff of Pm to predict individual's aging status from ROC curve analysis).

9. The method of claim 1, wherein, in the excess age calculation process, the individual's excess age is obtained by the weighted mean of every excess probability (Dm) to be predicted as the overage group (OAGm), and by applying an additional weight (Wm) to be applied, and
the weighted mean is calculated by Equation below:
$${Δ}_{i} = \frac{{\sum }_{m = x}^{y} {{m}^{⋆} ( {P}_{im} - {C}_{m} )}^{⋆} {w}_{m}}{y - x + 1}$$
$${\sum }_{x}^{y} {w}_{m} = 1$$
where, N: sample number i=1, 2, . . . , N,
Δi: weighted mean of (Pim−Cm),
Cm: cutoff value Cm obtained through age prediction probability calculation process
(cutoff of Pm to predict individual's aging status from ROC curve analysis), and
Wm: weight applied for model to predict CA≥m.

10. A system for generating a personalized biological age prediction model, the system comprising:
a checkup data collection means (110) configured to collect medical checkup data provided from a medical checkup system, and store and manage the medical checkup data in a data storage means (190);
a training data setting means (120) configured to determine valid training data from the checkup data provided from the checkup data collection means (110) according to a set training data reference age range (x to y) and checkup item information;
a binary logistic regression model generation means (130) configured to generate binary logistic regression models (Mx to My) for respective age units within the age range (x to y) set for the training data set by the training data setting means (120);
an age prediction probability calculation means (140) configured to calculate a probability (Pm) to be predicted as an overage group (OAGm) for each individual in the training data according to the binary logistic regression models generated by the binary logistic regression model generation means (130);
a cutoff extraction means (150) configured to set an underage group (UAGm) and the overage group (OAGm) as two-part response variables, set the probability (Pm) to be predicted as the over-age group (OAGm) as a predictor variable, and extract a cutoff (Cm) through ROC curve analysis;
an age prediction probability correction means (160) configured to apply (Pm−Cm) calculation to subtract the cutoff (Cm) from the probability (Pm), which is to be predicted as the overage group (OAGm) and calculated through the age prediction probability calculation means (140), calculate an excess probability (Dm) to be predicted as the individual overage group (OAGm), and correct the probability (Pm), which is to be predicted as the overage group (OAGm) and calculated by the age prediction probability calculation means (140);
an excess age calculation means (170) configured to obtain an individual's excess age by obtaining a weighted mean (Δi) for every excess probability (Dm) to be predicted as the overage group (OAGm) obtained through the age prediction probability correction means (160);
a biological age calculation means (180) configured to calculate a biological age from a chronological age by using the individual's excess age obtained through the excess age calculation means (170); and
the data storage means (190) configured to store and manage the medical checkup data collected from the checkup data collection means (110) and the training data set through the training data setting means (120).

11. The system of claim 10, further comprising:
a user setting means configured to provide a process enabling a user to retrieve and set the age range and the checkup item information of the training data setting means (120).

12. The system of claim 10 or 11, further comprising:
a user setting means configured to provide a process enabling the user to set condition information for determining the training data in the training data setting means (120).

13. The system of claim 12, where the condition information of the user setting means is male and female gender information.

14. The system of claim 10, wherein the binary logistic regression models (Mx to My) of the binary logistic regression model generation means (130) are generated for the respective age units by setting each age unit as one unit in the set age range, dividing the training data for each age unit into two groups of the underage group (UAGm) and the overage group (OAGm), setting the two groups of the underage group (UAGm) and the overage group (OAGm) as the response variables, and setting the training data as the predictor variable.

15. The system of claim 10 or 11, wherein the checkup item information of the training data setting means (120) is composed of health insurance checkup item data comprising:
physical examination indices such as body mass index, waist circumference, systolic blood pressure, and diastolic blood pressure; and
blood test indices such as three types of liver levels (i.e., AST, ALT, and γ-GTP), creatinine, three types of cholesterol (i.e., HDL, LDL, and TG), fasting blood glucose, and hemoglobin.

16. The system of claim 10, wherein the age prediction probability calculation means (140) performs calculating the probability (Pm) to be predicted as the overage group (OAGm) for each individual, who is a sample target, according to the binary logistic regression models by using Equation below:
$$p ( Y = {OAG}_{m} ) = \frac{exp ( {\sum }_{k = 0}^{p} {β}_{k} {X}_{k} )}{1 + exp ( {\sum }_{k = 0}^{p} {β}_{k} {X}_{k} )}$$
$$Define {P}_{im} = p ( {Y}_{i} = {OAG}_{m} )$$
where, Y: individual's aging status,
p(Y=OAGm): probability to be predicted as overage group OAGm,
Yi: i-th individual's aging status,
i=1, 2, . . . , : sample number,
m=26(x), 27, . . . , 75(y): (chronological age observed in training data),
CA: chronological age,
Xk: k-th independent variable,
βk: regression coefficient of k-th independent variable, and
p: number of independent variables.

17. The system of claim 10, wherein the excess age calculation means (170) obtains the individual's excess age by obtaining the weighted mean (Δi) for every probability (Dm) to be predicted as the overage group (OAGm) through Equation below:
$${Δ}_{i} = \frac{{\sum }_{m = x}^{y} {m}^{⋆} ( {P}_{im} - {C}_{m} )}{y - x + 1}$$
where, N: sample number i=1, 2, . . . , N,
Δi: weighted mean of (Pim−Cm), and
Cm: cutoff value Cm obtained through cutoff extraction means (150)
(cutoff of Pm to predict individual's aging status from ROC curve analysis).

18. The system of claim 10, wherein the excess age calculation means (170) obtains the individual's excess age by obtaining the weighted mean (Δi) for every probability (Dm) to be predicted as the overage group (OAGm) through Equation below:
$${Δ}_{i} = \frac{{\sum }_{m = x}^{y} {{m}^{⋆} ( {P}_{im} - {C}_{m} )}^{⋆} {w}_{m}}{y - x + 1}$$
$${\sum }_{x}^{y} {w}_{m} = 1$$
where, N: sample number i=1, 2, . . . , N,
Δi: weighted mean of (Pim−Cm),
Cm: cutoff value Cm obtained through cutoff extraction means (150)
(cutoff of Pm to predict individual's aging status from ROC curve analysis), and
Wm: weight applied for model to predict chronological age CA≥m.
